(19) United States
(12) Patent Application Publication
(10) Pub. No.: US 2023/0346973 A1
(43) Pub. Date: Nov. 2, 2023

(54) MICROORGANISM TARGETED NANOFORMULATIONS COMPRISING ANTIMICROBIAL COMPONENT(S)

(71) Applicant: YILDIZ TEKNIK UNIVERSITESI, Istanbul (TR)

(72) Inventors: Tulin ARASOGLU, Istanbul (TR); Serap DERMAN, Istanbul (TR); Murat TOPUZOGULLARI, Istanbul (TR); Mehmet OZBIL, Istanbul (TR); Bekir Mustafa YOGURTCU, Istanbul (TR)

(73) Assignee: YILDIZ TEKNIK UNIVERSITESI, Istnabul (TR)

(21) Appl. No.: 18/025,686

(22) PCT Filed: Dec. 7, 2020
(86) PCT No.: PCT/IB2020/001052

§ 371 Date: Mar. 10, 2023

(30) Foreign Application Priority Data
Sep. 10, 2020 (TR) 2020/14396

(51) Int. Cl. A61K 47/69; A61K 47/64; A61K 9/51

(52) U.S. Cl. CPC A61K 47/6937; A61K 47/64; A61K 9/5192; A61K 9/5153

(57) ABSTRACT

A nanostructure for lysis of pathogenic bacteria in mammals is provided. The nanostructure includes one or more antimicrobial components loaded into one or more carrier components forming a core portion. The nanostructure also includes one or more bacteriophage receptor binding proteins and/or one or more peptide sequences of the one or more bacteriophage receptor binding proteins attached on the core portion. A method for obtaining such a nanostructure is also provided.

### CROSS REFERENCE TO THE RELATED APPLICATIONS

This application is the national phase entry of International Application No. PCT/IB2020/001052, filed on Dec. 7, 2020, which is based upon and claims priority to Turkish Patent Application No. 2020/14396, filed on Sep. 10, 2020, the entire contents of which are incorporated herein by reference.

### TECHNICAL FIELD

The present invention relates to formulations containing one or more antimicrobial components. In particular, the present invention relates to microorganism-targeted nanoformulations containing one or more antimicrobial components.

### BACKGROUND

Pathogenic bacteria cause serious infections that threaten human health and many chronic diseases associated with biofilm formation (skin/mucosa and soft tissue infections such as folliculitis, impetigo, hidradenitis suppurativa, mastitis, wound infections, common pyodermia; bacteremia and endocarditis, pneumonia, bone/joint infections such as osteomyelitis, septic arthritis, septic bursitis, etc.). The treatment of infections using antibiotics was started with penicillin by A. Fleming in 1928, which was effectively used to control bacterial infections particularly during World War II, so lived its golden age until the 1940s. However, it has been detected in those years that the S. aureus strain is resistant against penicillin and the ensuing methicillin, which revealed that the treatment of infections should be carried out more consciously. Due to the antibiotic resistance resulting from the widespread and unconscious consumption of antibiotics, accompanied by a decrease in the rate of development of new agents, multidrug resistant, extensively drug resistant and pandrug-resistant bacteria strains have begun to be frequently identified and nowadays, fears of a “post-antibiotic era”—a time when many bacterial infections cannot be cured—have started to be expressed loudly. It is the resistant pathogenic bacteria that cause nosocomial infections, especially with a high mortality rate. According to the most recent data published by the World Health Organization, the hospital infection rate in our country is 13.4%. The 2015 UHESA (National Nosocomial Infections Surveillance Network) data report of the Public Health Agency of Turkey states that carbapenem-resistant Acinetobacter baumannii (68.9%) comes first in the distribution of antimicrobial-resistant pathogens among the bacteria that cause nosocomial infections, while methicillin-resistant coagulase-negative staphylococci (MRCNS) (67.84%) and methicillin-resistant S. aureus (MRSA) (44.8%) being the second and the third (Gözel 2016). Innovative approaches are required to prevent this situation.

Considering the difficulties in the traditional discovery and development of antimicrobial agents, it is now considered as a rational solution to eliminate antibiotic-resistant microorganisms using unconventional approaches. For this purpose, the following types of approaches are among the techniques that have started to be used commercially in recent years (Gao, W., Thamphiwatana, S., Angsantikul, P. and Zhang, L. 2014. “Nanoparticle approaches against bacterial infections.” Wiley Interdisciplinary Reviews: Nanomedicine Nanobiotechnology, 6(6), 532-547; Czaplewski, L., Bax, R., Clokie, M., Dawson, M., et al. 2016. “Alternatives to antibiotics—a pipeline portfolio review.” The Lancet infectious diseases, 16(2), 239-251.; Hauser, A. R., Mecsas, J. and Moir, D. T. 2016. “Beyond antibiotics: new therapeutic approaches for bacterial infections.” Clinical Infectious Diseases, 63(1), 89-95.; Hussain, S., Joo, J., Kang, J., Kim, B., et al. 2018. “Antibiotic-loaded nanoparticles targeted to the site of infection enhance antibacterial efficacy.” Nature biomedical engineering, 2(2), 95; Altamirano, F. L. G., Jeremy J 2019. “Phage therapy in the postantibiotic era.” Clinical microbiology reviews, 32(2), e00066-00018):
use of antibodies,
the mechanism of action of probiotics and the use thereof in combination with antibiotics and/or other alternatives,
use of phage lysines utilized by bacteriophages to destroy the cell wall of a target bacterium in combination with antibiotics and/or other alternatives,
direct use of wild-type bacteriophages that infect and kill bacteria,
genetically producing phages having new properties for therapeutic use (host diversity, resistance development, rapid intracellular elimination after administration, dose selection),
applications such as phenyl butyrate, vitamin D, oral bacterial extracts to provide immune stimulation along with antibiotic therapy,
production of vaccines for new targets,
antimicrobial peptides with bactericidal effects, low target-based resistance, and low immunogenicity,
use of targeted peptides that will allow direct interaction between the drug and the microorganism,
use of antibiotic cocktails that create synergistic effects,
increasing the effectiveness of antibiotics with nanotechnological approaches and reducing resistance.

Bacteriophages themselves (wild-type lytic phages) or lysine enzymes are currently used for antibacterial therapy. These practices can be summarized in paragraphs (a) and (b) below:
a) Lytic bacteriophage therapy begins with an attachment of a phage to the host bacterium. After the attachment, the phage discharges the genetic material into the host cell and then takes over the replication mechanism of the host bacterial cell. Inside the cell, “daughter” phages are produced and then the cell is lysed. Thanks to this mechanism, lytic bacteriophage is introduced into many antibiotic resistant bacteria so that they are lysed.
b) Bacteriophage lysines may vary from bacteria to bacteria. Lysines break down the peptidoglycan structure of the cell membrane in the target bacteria, causing the cell to be lysed. As an example, in studies conducted on S. aureus, it is determined by in vitro and in vivo studies that a lysine enzyme is produced recombinantly and exhibits antibacterial activity on the methicillin-resistant S. aureus. It has also been found that this enzyme is combined with antibiotics and has an antibacterial synergistic effect on bacteria. It is also known in the literature that gentamicin and lysine enzyme have a synergistic effect and decrease the MIC value.

For all these reasons, there is still a need for further improvement of the formulations that exhibit high efficiency in the treatment of infections.

The main object of the invention is to provide solutions to the problems mentioned in the prior art.

### SUMMARY

The present invention provides a nanostructure for lysis of a pathogenic bacteria in mammals. Said nanostructure comprises one or more antimicrobial components loaded into one or more carrier components forming a core portion. The nanostructure also comprises one or more bacteriophage receptor binding proteins and/or one or more peptide sequences of the said bacteriophage receptor binding protein, attached on the said core portion.

In one embodiment of the invention, the nanostructure comprises one or more bacteriophage receptor binding proteins attached on the said core portion, in which case it can be considered that the nanostructure does not contain one or more peptide sequences alone.

In an alternative embodiment of the invention, the nanostructure comprises one or more peptide sequences of the said one or more bacteriophage receptor binding proteins attached on the said core portion, in which case it can be considered that the nanostructure does not contain the bacteriophage receptor binding protein as a whole.

In another alternative embodiment of the invention, the nanostructure may comprise said one or more bacteriophage receptor binding proteins, and one or more peptide sequences of the said one or more bacteriophage receptor binding proteins, together, as attached on the said core portion.

Any version of the nanostructure of the invention may comprise, as an antimicrobial component, one or more of the following: one or more antibiotics, one or more antimicrobial peptides, one or more secondary metabolites, or one or more antimicrobial agents. In this respect, any version of the nanostructure may comprise one or more elements selected from the following: one or more antibiotics selected from vancomycin, oxacillin, and gentamicin; one or more antimicrobial peptides selected from magainin, alamethicin, and pexiganan; one or more secondary metabolites selected from juglone, quercetin, and catechin, as an antimicrobial component. The antimicrobial agent used in any version of the nanostructure of the invention may also be one or more antimicrobial agents known in the relevant art to be suitable for antimicrobial purposes.

Any version of the nanostructure of the invention may comprise, as a carrier component, one or more elements selected from the following: one or more proteins, one or more polymers, one or more lipids. Alternatively, or additionally, the carrier component may comprise one or more carrier molecules known in the art for this purpose. In this respect, any version of the nanostructure may comprise, as a carrier component, one or more elements selected from the following: one or more proteins selected from bovine serum albumin, human serum albumin, and ovalbumin; one or more polymers selected from PLGA, PLMA, and PCL; one or more lipids selected from 1,2-dilauroyl-sn-glycero-3-phosphocholine, 1,2-dimyristoyl-sn-glycero-3-phosphocholine, 1,2-dioleoyl-sn-glycero-3-phosphocholine.

Any version of the nanostructure of the invention may preferably have a diameter less than 500 nm and a PDI value less than 0.5.

The present invention also discloses a method for producing any version of the nanostructure of the invention. In this context, the method is a method for producing a nanostructure for lysis of a pathogenic bacteria in mammals, comprising one or more antimicrobial components loaded into one or more carrier components forming a core portion, also comprising one or more bacteriophage receptor binding proteins and/or one or more peptide sequences of the said bacteriophage receptor binding protein, attached on the said core portion. Said method comprises the following steps of:
obtaining said one or more bacteriophage receptor binding proteins by any one of the methods/technologies known in the relevant art to be suitable for use in the production of RBP (1), such as recombinant technology, electrophoretic methods, chromatographic methods, and centrifugation-based methods; and/or bioinformatically detecting one or more peptide sequences located in an active binding site of one or more bacteriophage receptor binding proteins by molecular docking and simulation, and synthesizing the peptide sequence (e.g., using any one of the methods known in the relevant art; e.g. microwave-assisted solid phase synthesis, solution-phase synthesis, etc.);
synthesizing said one or more carrier components, conjugating said bacteriophage receptor binding protein and/or said one or more peptide sequences located in the active binding site of one or more bacteriophage receptor binding proteins on the said carrier component, so that said carrier component is located in a core portion;
loading said one or more antimicrobial components into the said one or more carrier components.

### BRIEF DESCRIPTION OF THE DRAWINGS

The drawings, whose brief description is provided below are just given for better understanding of the present invention and as such, are not intended to determine the scope of the claimed subject matter, in the absence of the description.

FIG. 1 is a schematic illustration representing an exemplary embodiment of the formulation of the present invention. Here, the nanoformulation/nanostructure is provided with RBPs.

FIG. 2 is a schematic illustration representing another exemplary embodiment of the formulation of the present invention. Here, the nanoformulation/nanostructure is provided with peptide sequences of RBPs.

FIG. 3 is a schematic illustration representing another exemplary embodiment of the formulation of the present invention. Here, the nanoformulation/nanostructure is provided with peptide sequences of RBPs, in addition to RBPs.

### DETAILED DESCRIPTION OF THE EMBODIMENTS

The design of the nanoformulations according to the present invention is aimed at obtaining nanoformulations targeted to the relevant organisms that can be effective in the treatment of diseases caused by bacteria (due to increases in the distribution of resistant pathogens in the hospital-acquired infections, for example especially in Turkey, because of data accessibility, and mortality rates) and comprising antimicrobial component(s), in order to provide unique therapeutic agents.

The present improvement is directed to a nanostructure (5) comprising a bacteriophage receptor binding protein (1) (RBP), and, in addition or alternatively, a peptide sequence (2) of the bacteriophage receptor binding protein. Said RBP (1) and the peptide sequence (2) of the RBP, respectively, may be considered as corresponding to the following:
1) one or more proteins (RBP (1)), and
2) one or more molecules in peptide structure (peptide sequence (2)),
   which provide bacteria targeting and are chemically bonded to the nanoformulation that is produced, in order to allow the antimicrobial component(s) contained therein to directly reach a targeted bacterium.

The nanostructure (5) of the invention may be considered as a nanoformulation and may also be referred to as “nanoformulation” throughout the description.

In the present description, the term “RBP (1)” can be considered as corresponding to one or more RBPs (1) of the type that will cause bacteriophage binding. The term “peptide sequence (2) of RBP (1)” is that one or more RBPs (1) conforming to the description in the previous sentence correspond to the peptide sequence (2) that exhibits receptor-ligand interaction and appears to have a high (preferably highest) score as a result of molecular docking and simulations.

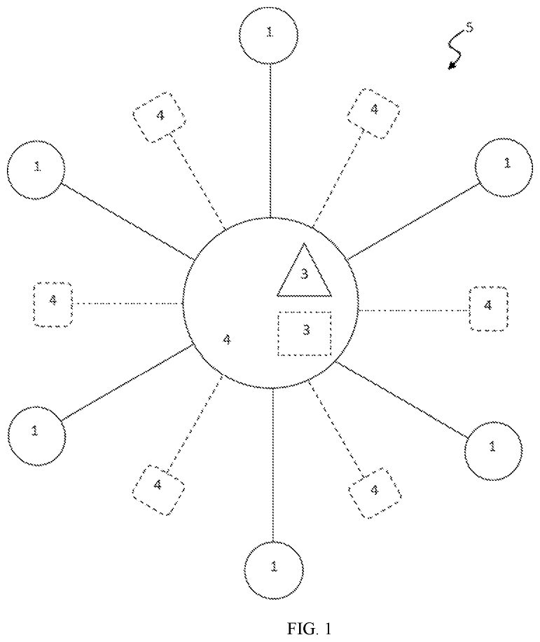

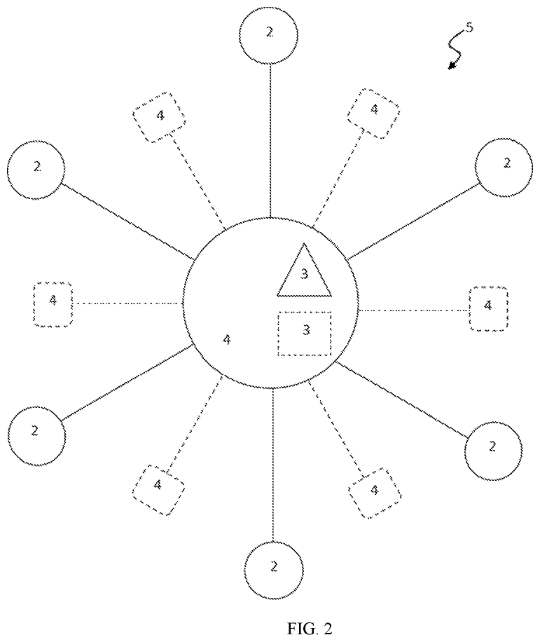

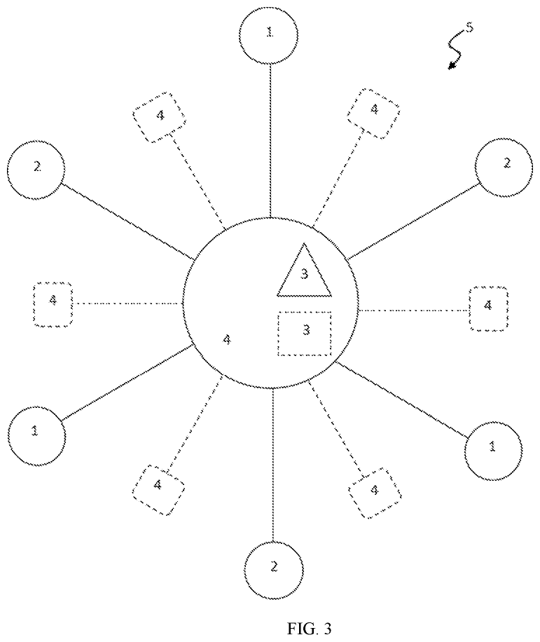

In a preferred embodiment of the invention, the nanostructure (5) may comprise RBP (1) alone on the carrier component (4) loaded with the antimicrobial component (3) (see, the exemplary embodiment in FIG. 1), or alternative to the RBP (1), may comprise said peptide sequence (2) (see, the exemplary embodiment in FIG. 2); or may comprise said peptide sequence (2) together with RBP (1) (see exemplary embodiment in FIG. 3). In a preferred embodiment of the invention, the nanostructure (5) comprises said peptide sequence (2) together with the RBP (1).

The function of both RBP (1) and the peptide sequence (2) of the bacteriophage receptor binding protein is to directly target the bacterial cell. An RBP (1) or a peptide sequence (2) thereof used in the invention may be located at a distal end of the nanostructure (5) with respect to a base plate (i.e., a core portion of the carrier component (4), that is, a core portion in the nanostructure (5)). Thus, by interacting with proteins, saccharides, and organelles in units such as peptidoglycan, wall teichoic acids (WTA) and lipoteichoic acid located on a wall surface of a bacterial cell, it induces inactivation of the bacteria by means of the antimicrobial component(s) (3) in the nanostructure (5).

The present invention may also include process steps for obtaining RBP (1) using any one of the methods within the general knowledge of the relevant technical field. For an exemplary case where RBP (1) is obtained by recombinant DNA technology, said method may include the following steps, respectively:
obtaining the gene region of the RBP (1) and enabling the amplification of the gene (by any one of the methods within the general knowledge in the relevant technical field),
(here: recombinant) transfer of the bacteriophage receptor binding protein (1) to a host (e.g., E coli) and expression thereof (by any one of the methods within the general knowledge in the relevant art);
(here: recombinant) purification of RBP (1) and/or the peptide sequence (2) thereof (by any one of the methods within the general knowledge in the relevant technical field): for example, synthesis and purification of the peptide sequence (or peptide sequences) (2) of RBP (1) exhibiting the receptor-ligand interaction and having the highest score from the molecular docking and simulations (by any one of the methods within the general knowledge of the relevant technical field (e.g., microwave-assisted solid phase synthesis, solution-phase synthesis, etc.).

As an indication of industrial applicability: e.g., cyclic 9-amino-acid peptide CARGGLKSC (CARG) (Hussain, S., Joo, J., Kang, J., Kim, B., et al. 2018. “Antibiotic-loaded nanoparticles targeted to the site of infection enhance antibacterial efficacy.” Nature biomedical engineering, 2(2), 95), for example may be used as a targeting peptide (2) for S. aureus. New peptide sequences (2) suitable for targeting both S. aureus and other bacteria may be bioinformatically detected by molecular docking and simulations. Peptide sequences (2) may be synthesized and purified by methods known in the literature (e.g., microwave-assisted solid phase peptide synthesis method: Derman, S., Kizilbey, K., Mansuroglu, B. and Mustafaeva, Z. 2014. “Synthesis and characterization of canine parvovirus (CPV) VP2 W-7L-20 synthetic peptide for synthetic vaccine.” Fresen Environ Bull, 23 (2A), 558-566).

In addition, one or more antimicrobial components (3) are included in the nanoformulation of the present invention. The function of this element is to ensure the lysis of targeted bacteria. Examples of antimicrobial components (3) may include antibiotics (vancomycin, oxacillin, etc.), antimicrobial peptides (magainins, alamethicin, pexiganan, etc.), and secondary metabolites (juglone, quercetin, catechin, etc.). Said one or more antimicrobial components (3) correspond to one or more molecules that have a natural or synthetic antibacterial action leading to lysis of a targeted bacteria.

In the literature, there is no publication describing bacteria targeting through conjugation of a nanosystem/nanoformulation/nanostructure (1) loaded with one or more antimicrobial components (3) (e.g., vancomycin, oxacillin) with one or more RBP (1) and/or peptide sequences (2).

In addition to the nanostructure (5) of the present invention may also include one or more carrier components (4), which are known to be suitable for use in pharmaceutical production, and selected from proteins, lipids, and polymers. The carrier component (4) allows said one or more antimicrobial components (3) to be transported within the nanostructure (5). Because of their widespread use in the state of the art, the following can be listed as examples of materials suitable for use as a carrier component (4):
proteins such as bovine serum albumin, human serum albumin, ovalbumin;
polymers such as PLGA (poly (lactide-co-glycolide)), PLMA (poly(malic acid)), PCL (poly (caprolactone));
lipids such as 1,2-dilauroyl-sn-glycero-3-phosphocholine, 1,2-dimyristoyl-sn-glycero-3-phosphocholine, 1,2-dioleoyl-sn-glycero-3-phosphocholine.

When the nanostructure (5) is formed, the carrier component (4) can be located in the center of the nanostructure (5) by being loaded into one or more antimicrobial components (3) described above; alternatively, or additionally, it may extend outwards from the center.

By suitably combining the above elements, a bacteria-targeted nanostructure (5) comprising one or more antimicrobial component(s) (3) is achieved with the present invention.

It is preferred that the nanostructure (5) has a diameter (size) less than 500 nm and a PDI value less than 0.5. This makes one or more of the following simultaneously possible: protection of the antimicrobial component(s) (3) from biological degradation, thus allowing the components to remain in systematic circulation for a long period of time; performing controlled releases of the antimicrobial component(s) (3); and carrying the RBP (1) and/or the peptide sequence (2) directly targeting the bacterial cell on its surface.

A suggested way of obtaining the nanostructures (5) of the invention can be described as follows:

Providing one or more RBPs (1) and/or one or more peptide sequences (2) thereof. Providing one or more antimicrobial components (3) (by any one of the methods within the general knowledge in the relevant technical field, e.g., single emulsion, multiple emulsion, solvent coaservation, emulsification, etc.,). Providing one or more carrier components (4) to form a core portion of the nanostructure (5) to be produced (by any one of the methods within the general knowledge in the relevant technical field). Loading/attaching said one or more antimicrobial components (3) into the carrier component(s) (4) constituting the said core portion (by any one of the methods within the general knowledge in the relevant technical field). Binding one or more RBPs (1) and/or one or more peptide sequences (2) thereof that are provided, on the core portion loaded with the antimicrobial component(s) (3) (by any one of the methods within the general knowledge in the relevant technical field, e.g., bioconjugation methods).

The targeting phase of the invention (which may be considered as a first step) can be accomplished using the following strategy(s);
providing RBP (1), or obtaining thereof by any one of the methods known to be suitable in the relevant art such as recombinant technology, electrophoretic, chromatographic, centrifugation, etc.; and/or detecting bioinformatically of the peptide sequences (2) located in the active binding site of the RBP (1), by molecular docking and simulations, and synthesis thereof by any one of the methods known in the relevant art such as microwave-assisted solid phase synthesis or solution-phase synthesis.

Then, nanostructures (5) that are loaded and targeted, and containing the antimicrobial component(s) (3) may be obtained (which may be considered as a second step of the method). As an example, it is possible to perform bioinformatical detection and synthetic production of the peptide sequences (2) of RBP (1) that bind to a binding site on the cell wall of a bacterium (e.g., S. aureus cell) with the highest binding energy. For this purpose, a loaded carrier component (4) (e.g., a micellar form biopolymer) containing the antimicrobial component(s) (3) is first produced. The surface of the said carrier component (4) obtained to form a core portion is modified by conjugation of the bacteriophage receptor binding protein (RBP (1)) and/or peptide sequences (2) thereof. Thus, direct targeting may be achieved (in this example, direct targeting of the bacterium S. aureus is achieved).

Thus, nanostructures (5) containing antimicrobial component(s) (3) are produced. With the antimicrobial component(s) (3), the nanostructure (5) exhibits a synergistic effect.

Thanks to the improvement of the present invention, the potential of both RBP (1) and the peptide sequences (2) thereof to be used for diagnostic and therapeutic purposes is ascertained.

Thanks to the nanostructure (5) of the present invention, contrary to the paragraph (a) in the “background art” section: the RBP (1) and/or the peptide sequence (2) thereof causing the binding of the bacteriophage can be used for targeting purposes. Thus, instead of delivering a living phage (virus) to the host, it is possible for the antimicrobial component(s) (3) constrained in the nanostructure (5) to directly lyse the bacterial cell.

Furthermore, thanks to the nanostructure (5) of the present invention, contrary to the paragraph (b) in the “background art” section: The RBP (1) and/or the peptide sequence (2) thereof used in targeting can bind to wall teichoic acid (WTA) units in a wall of a targeted cell. WTA are phosphate-rich anionic glycopolymers covalently bonded to peptidoglycan in Gram-positive bacteria. (L Li, X., Koc, C., Kuhner, P., Stierhof, Y.-D., et al. 2016. “An essential role for the baseplate protein Gp45 in phage adsorption to Staphylococcus aureus.” Scientific reports, 6, 26455). By obtaining the bacteriophage receptor binding protein (RBP (1)) or synthetically producing the peptide sequence (2) thereof with the highest binding energy, the resulting product is conjugated on a core portion to obtain the nanostructure (5), as a result of which direct bacteria targeting is rendered possible. Thus, using the antimicrobial component(s) (3) together with the phage receptor binding protein/peptide (i.e., RBP (1) and/or the peptide sequence (2) thereof) located on the surface of the encapsulated nanostructure (5), the relevant bacterial strains are directly targeted in this study, so that an effect just like the phage therapy may be created, and the effectiveness of the bacterial lysis may be further increased.

Each stage of the method for producing the nanostructure (5) of the invention is suitable for production with high quantity and capacity. The subject-matter of the invention is particularly specific for industrial establishments serving in the field of health and pharmacy. A method of administration of the nanostructures (5) loaded with antimicrobial component(s) (3) and targeted to bacteria, which is the final product planned to be obtained, in infectious diseases, is the same as the administration of antibiotics (e.g., vancomycin, oxacillin) (e.g., IV-intravenous, IM-intramuscular, etc.) For example, in the presence of antibiotic resistance in a patient hospitalized in an intensive care unit with S. aureus infection; the nanostructure (5) targeted against the respective bacterium with the peptide/recombinant protein (RBP (1)) attached thereon and/or the peptide sequence (2) thereof, and containing one or more antimicrobial components (3) (e.g. antibiotics such as vancomycin, ampicillin, oxacillin, etc.; antimicrobial peptides such as magainines, alamethicin, pexiganan, etc.; secondary peptides such as juglone, quercetin, catechin, etc.) is administered, which then selectively bind to the wall teichoic acid (WTA) units on the surface of S. aureus, thereby resulting in the treatment of the infection by rapidly eradicating the relevant bacteria. At the same time, the disadvantages of the antimicrobial component(s) (3) with low water solubility or short half-life in the biological system can be eliminated.

Thanks to the inventive improvement, the deficiencies of the prior art have been eliminated and the aforementioned problems have been solved.

### REFERENCE NUMERALS

1 RBP
2 RBP peptide sequence
3 antimicrobial component(s)
4 carrier component(s)
5 nanoformulation (or nanostructure)

What is claimed is:

1. A nanostructure for lysis of a-pathogenic bacteria in mammals, comprising
one or more antimicrobial components loaded into one or more carrier components forming a core portion of the nanostructure;
one or more bacteriophage receptor binding proteins and/or one or more peptide sequences of the one or more bacteriophage receptor binding proteins, attached on the core portion.

2. The nanostructure according to claim 1, comprising the one or more bacteriophage receptor binding proteins attached on the core portion.

3. The nanostructure according to claim 1, comprising the one or more peptide sequences of the one or more bacteriophage receptor binding proteins attached on the core portion.

4. The nanostructure according to claim 1, comprising the one or more bacteriophage receptor binding proteins and the one or more peptide sequences of the one or more bacteriophage receptor binding proteins, attached on the score portion.

5. The nanostructure according to claim 1, wherein the one or more antimicrobial components are one or more selected from one or more antibiotics, one or more antimicrobial peptides, and one or more secondary metabolites.

6. The nanostructure according to claim 5, wherein the one or more antibiotics are selected from vancomycin and oxacillin; the one or more antimicrobial peptides are selected from magainin, alamethicin, and pexiganan; the one or more secondary metabolites are selected from juglone, quercetin, and catechin.

7. The nanostructure according to claim 1, wherein the one or more carrier components are one or more selected from one or more proteins, one or more polymers, and one or more lipids.

8. The nanostructure according to claim 7, wherein the one or more proteins are selected from bovine serum albumin, human serum albumin, and ovalbumin; the one or more polymers are selected from PLGA, PLMA, and PCL; the one or more lipids are selected from 1,2-dilauroyl-sn-glycero-3-phosphocholine, 1,2-dimyristoyl-sn-glycero-3-phosphocholine, and 1,2-dioleoyl-sn-glycero-3-phosphocholine.

9. The nanostructure according to claim 1, wherein a diameter of the nanostructure is less than 500 nm and a PDI value of the nanostructure is less than 0.5.

10. A method for producing a nanostructure for lysis of pathogenic bacteria in mammals, the nanostructure comprising one or more antimicrobial components loaded into one or more carrier components forming a core portion; one or more bacteriophage receptor binding proteins and/or one or more peptide sequences of the one or more bacteriophage receptor binding proteins attached on the core portion, the method comprising the steps of:
obtaining the one or more bacteriophage receptor binding proteins; and/or bioinformatically detecting the one or more peptide sequences located in an active binding site of the one or more bacteriophage receptor binding proteins by molecular docking and simulation, and synthesizing the one or more peptide sequences;
producing the one or more carrier components loaded with the one or more antimicrobial components, conjugating the one or more bacteriophage receptor binding proteins and/or the one or more peptide sequences located in the active binding site of the one or more bacteriophage receptor binding proteins on the one or more carrier components, so that the one or more carrier components are located in the core portion.

11. The method according to claim 10, wherein the one or more antimicrobial components are is selected from one or more antibiotics, one or more antimicrobial peptides, and one or more secondary metabolites.

12. The method according to claim 11, wherein the one or more antibiotics are selected from vancomycin and oxacillin; the one or more antimicrobial peptides are selected from magainin, alamethicin, and pexiganan; the one or more secondary metabolites are selected from juglone, quercetin, and catechin.

13. The method according to claim 10, wherein the one or more carrier components are selected from one or more proteins, one or more polymers, and one or more lipids.

14. The method according to claim 13, wherein the one or more proteins are selected from bovine serum albumin, human serum albumin, and ovalbumin; the one or more polymers are selected from PLGA, PLMA, and PCL; the one or more lipids are selected from 1,2-dilauroyl-sn-glycero-3-phosphocholine, 1,2-dimyristoyl-sn-glycero-3-phosphocholine, and 1,2-dioleoyl-sn-glycero-3-phosphocholine.

15. The nanostructure according to claim 2, wherein the one or more antimicrobial components are one or more selected from one or more antibiotics, one or more antimicrobial peptides, and one or more secondary metabolites.

16. The nanostructure according to claim 3, wherein the one or more antimicrobial components are one or more selected from one or more antibiotics, one or more antimicrobial peptides, and one or more secondary metabolites.

17. The nanostructure according to claim 4, wherein the one or more antimicrobial components are one or more selected from one or more antibiotics, one or more antimicrobial peptides, and one or more secondary metabolites.

18. The nanostructure according to claim 2, wherein the one or more carrier components are one or more selected from one or more proteins, one or more polymers, and one or more lipids.

19. The nanostructure according to claim 3, wherein the one or more carrier components are one or more selected from one or more proteins, one or more polymers, and one or more lipids.

20. The nanostructure according to claim 4, wherein the one or more carrier components are one or more selected from one or more proteins, one or more polymers, and one or more lipids.
